(19) United States
(12) Patent Application Publication
(10) Pub. No.: US 2008/0076970 A1
(43) Pub. Date: Mar. 27, 2008

(54) FLUID MANAGEMENT MEASUREMENT MODULE

(76) Inventors: Mike Foulis, Madison, WI (US); Malla Sarajarvi, Porvoo (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/535,318

(22) Filed: Sep. 26, 2006

(51) Int. Cl. A61B 5/00

(52) U.S. Cl. 600/300

(57) ABSTRACT

A system and method for monitoring and analyzing patient urinary output and a plurality of additional patient physiological parameters. The system comprises a plurality of patient connections and monitoring apparatus for monitoring a plurality of patient physiological parameters. The system further comprises a processor programmed to apply at least one analysis criterion to at least a signal indicative of patient urinary output. The processor further applies additionally more complex analysis criterion to a combination of patient urinary output data and at least one additional patient physiological parameter to provide an automated diagnosis of patient condition.

### FIELD OF THE INVENTION

The field of the invention is related to patient monitoring systems, specifically monitoring systems integrating a plurality of measurements of patient physiological data.

### BACKGROUND OF THE INVENTION

The purpose of patient monitoring is to acquire a detailed picture of the condition of a patient. This picture is developed by viewing the values of measured physiological parameters that represent particular aspects of bodily functions. Physiological parameter values may also be viewed in combination to determine relationships between parameter values. Examples of commonly monitored physiological parameters include parameters that measure cardiac function such as ECG, blood pressure, pulse oximetry (SPO2); respiratory function such as respiration rate, exhalation composition, functional residual capacity (FRC); and other physiological parameters such as temperature, brain activity (EEG), and muscle activity (EMG).

Each of these physiological parameters provides a clinician with a useful tool for determining the condition of a specific aspect of a patient's health. However, one physiological parameter that is often overlooked in a clinical context is the urine output of the patient. The urine output of the patient is useful as a physiological parameter in patient monitoring depending upon the level of urinary output analysis. Patient urine output can be an indication of basic patient health, or can be utilized as a component of a more detailed analysis of the patient condition. Presently, there is a lack of urine output monitoring in current automated patient monitoring systems.

Traditionally, urine output is monitored manually by a clinician checking a urine collection receptacle at various time intervals and recording the amount of urine output collected from the patient. Automated urine output monitoring, as taught by Corbitt et al U.S. Pat. No. 4,449,538, is known in the art. Automated urine output monitoring systems, such as that taught in the '538 patent, are limited in that they do not look at urine output as a physiological parameter on its own, but rather as merely a component of the parameter of total body fluid output, and fail to combine the urine output data with diverse types of physiological data to achieve a new picture of patient condition.

Other patient monitoring systems, such as that disclosed in U.S. Pat. No. 5,687,717 to Halpern et al, teach of a modular patient monitoring system that comprises separate modules for the collection and display of patient physiological parameters. However, systems as taught in the '717 patent are limited in that they do not combine the collected physiological data, including the urine output data, into an analysis of multiple patient physiological parameters to aid a clinician in analyzing patient condition.

Therefore, it is desirable in the field of patient monitoring systems for a system that enables the collection of urine output, the automated detection of the volume of the urine output, and the improved use of the volume of urinary output as a physiological parameter in a patient monitoring system.

### SUMMARY OF THE INVENTION

The present invention provides a system for patient monitoring that comprises data that is representative of the urinary output of a patient.

In an embodiment of the invention, the patient monitoring system utilizes the urinary output of the patient as a physiological parameter on its own as a basic indication and determination of patient condition.

In a further embodiment of the invention, the urinary output of the patient is utilized in conjunction with other monitored patient physiological parameters to provide a more complex determination of patient condition. In a more specific embodiment of the present invention, the system utilizes a rules engine to combine measured values of patient physiological parameters including urinary output in formulating a determination of patient condition.

In another embodiment of the invention, the urinary output of the patient is automatedly monitored as it is collected from the patient and the collected urinary output data is sent to the monitoring system for analysis.

In a still further embodiment of the invention, patient data is recorded after each type of urinary output analysis has been performed.

In still another embodiment of the invention, a system of clinician alarms and/or indicators is established such that the clinician is notified when predefined urinary output conditions have been met, or have failed to have been met.

### BRIEF DESCRIPTION OF THE DRAWINGS

The drawings illustrate the best mode contemplated of carrying out the invention. In the drawings:

FIG. 1 depicts a schematic diagram of an embodiment of the present invention; and

FIG. 2 depicts a flow chart of the operation of an embodiment of the present invention.

### DETAILED DESCRIPTION OF THE INVENTION

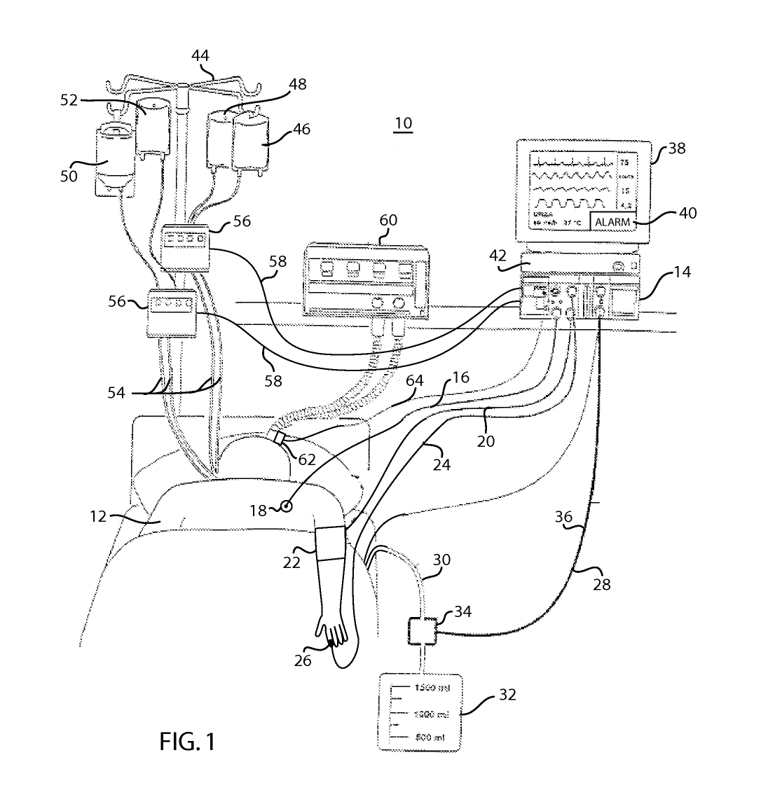

FIG. 1 depicts a schematic diagram of the patient monitoring system 10 of the present invention. In the patient monitoring system 10, a patient 12 is having his or her condition monitored by a variety of physiological monitoring systems that are shown as modules in a monitoring unit 14. It is understood that the monitoring unit 14 may comprise a plurality of patient physiological parameter monitoring units as disclosed herein; however, the monitoring unit 14 may also comprise a CPU (not pictured) that receives and processes physiological data from a plurality of physiological parameter monitoring units. Furthermore, the physiological parameter monitoring units may be remotely located. These remotely located physiological parameter monitoring units may transmit the physiological data to the monitoring unit 14 via wired or wireless communication systems.

In one embodiment, as depicted in FIG. 1, the monitoring unit 14 comprises the patient physiological parameter monitoring systems of electrocardiography (ECG) 16 as measured by one or more electrodes 18, blood pressure 20 as measured by a blood pressure cuff 22, pulse oximetry (SₚO₂) 24 as measured by a pulse oximeter 26, and urinary output 28, as further described herein. It is further contemplated within the scope of the present invention that many other physiological parameters may be utilized in the system within the scope of the present invention. The physiological parameter monitoring systems as described are utilized for merely exemplary purposes and are not meant to be limiting upon the scope of the present invention.

The urinary output 28 of the patient 12 may be monitored in a variety of ways. In an embodiment of the present invention, the patient has been catheterized with a catheter 30, such as a Foley catheter, that is inserted into the bladder of the patient 12. The catheter 30 removes the urine from the patient's bladder and directs the urine from the patient into a collection receptacle 32. Once collected, the urine may be disposed of, or sent to a medical laboratory for analysis of the content of the urine.

In an embodiment of the present invention, a sensor 34 is disposed along the path of the catheter 30 prior to the collection receptacle 32. The sensor 34 monitors the flow of the urine passing through the catheter 30 and relays the urinary output data 28 via a communication line 36.

It is understood within the present invention that the sensor 34 may comprise any number of types of sensors suitable for monitoring the movement of a liquid in a catheter. Typical sensors that may be used in conjunction with the present invention, as described in a non-limiting fashion, may include a hot wire anemometer, a laser-doppler anemometer, a venturi meter, an electromagnetic flow meter, an ultrasonic flow meter, or a turbine flow meter; however, it is understood that any other suitable sensor not explicitly herein mentioned may be used in accordance with the present invention. Furthermore, in embodiments of the present invention, the sensor 34 may be disposed at any point along the catheter 30, or may be disposed within the collection receptacle 32. The specific location of the sensor 34 is immaterial to the present invention, provided that the sensor 34 is positioned such that the sensor 34 can monitor the urine output of the patient 12.

It is understood that in embodiments of the present invention, the data measured by the sensor 34 may be flow rate data and this flow rate data may be combined with the diameter of the catheter 30 at which the flow rate is monitored such that over time a determination of volume of urine output is determined. Alternatively, the determination of urine output volume may be achieved by an optical sensor (not depicted) located in the collection receptacle 32 such that the volume of urine output may be monitored by optically determining the volume of urine collected in the collection receptacle 32.

The urinary output data 28 is sent along communications line 36 from the sensor 34 to the monitoring unit 14. The monitoring unit 14 comprises a CPU (not depicted) that is programmed to process the physiological data received by the monitoring unit 14, specifically the urinary output data 28. The CPU processes the received physiological data, some of the processing being in accordance with the method of the present invention, and displays graphical representations of the physiological data on a display 38. If the physiological data received is outside of a predefined acceptable data range, or if an alarm condition as in accordance with the present invention has been met, the CPU of the monitoring unit 14 may initiate an alarm signal. The alarm signal may be a visual alarm 40 displayed on the display 38, or the alarm may be an audible alarm that may emanate from a speaker 42. The speaker 42 may be associated with the monitoring unit 14, the display 38, or some other audio communications system. It is further understood that an audio or a visual alarm may be activated that is part of an alternative clinician communications platform, such as a PA system, cellular communications, or electronic clinician message boards.

In an embodiment of the monitoring system 10 of the present invention, the patient 12 is also receiving medical support from a variety of modular medical devices or systems. This modular medical support may comprise the intravenous (IV) delivery of fluid-based medical treatment from an IV delivery system 44. The IVs in the IV delivery system 44 may comprise a drug IV 46, an anesthetic IV 48, a fluid or saline IV 50, or a nutritional IV 52. The IV delivery system 44 delivers the content of the IVs to the patient 12 via a plurality of catheters 54. The IV delivery system 44 further comprises at least one IV volume monitor 56. In an embodiment of the present invention, the IV volume monitor 56 is a drip counter that monitors the number of equal volume drips from the IV bag into the catheters 54, thus monitoring the volume of the IV substance that is delivered to the patient 12. Alternatively, the IV volume monitor 56 may comprise a flow sensor associated with the IV catheter 54 such that the volume and flow rate of the IV substance that is delivered to the patient 12 may be monitored in a similar fashion as the patient urine output is monitored. The monitored IV delivery information is transmitted from the IV volume monitor 56 to the monitoring unit 14 via a communications line 58.

In an embodiment of the present invention, the patient 12 is receiving ventilatory support from a mechanical ventilator 60. The ventilator 60 supplies the patient 12 with respiratory support in the form of mechanical ventilation or mechanical ventilatory assistance. Additionally, the ventilator 60 may provide the patient with more complex forms of mechanical ventilatory assistance or therapy such as the application of positive end expiratory pressure (PEEP). Furthermore, the ventilator 60 may also provide the patient with a source of supplemental medical gas (not depicted). The supplemental medical gas may comprise a non-air gas such as additional oxygen or an anesthetic agent. A patient monitoring system 10 of the present invention may comprise a gas analysis module 62. The gas analysis module 62 may comprise the ability to sample and analyze either the gas delivered to the patient, the gas expired by the patient, or both to determine the composition of these gases. The analysis of the composition of gases supplied to the patient and expired by the patient may provide indications of patient metabolism, cardiovascular health, and pulmonary health. The gas analysis module 62 may send physiological data representative of the gas analysis to the monitoring unit 14 via a communications line 64.

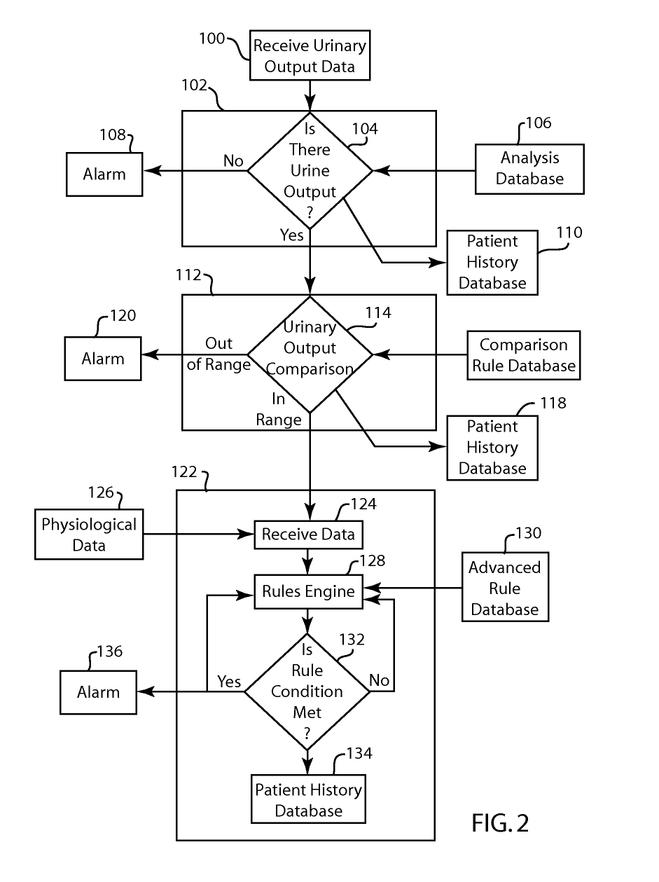

FIG. 2 is a flow chart of an embodiment of the operation of a monitoring unit 14 of the present invention. The monitoring unit 14 first receives urine output data 28 at step 100. As previously described, the urine output data 28 may comprise urine output information such as the flow rate, the urine volume, and time indications. As an embodiment of the present invention, the urine output data will be represented by the urine output volume and will herein be used in an exemplary fashion; however, it is understood that urine output flow may be similarly used in an embodiment of the present invention.

In an embodiment of the present invention, the urine output data is processed in a series of groups of analysis criterion or rules in order to pull the desired patient conditions from the urine output physiological data. The urine output data first undergoes a basic urinary output interpretation 102. There is an inherent relationship between the existence of urine output and basic bodily function. In some medical situations, especially post-operative patient monitoring, it is desirable to first determine whether a patient is producing any urine output at all within a specified time period after the operation. Therefore, at step 104, the monitoring unit 14 monitors the urine output data and determines whether or not there is a urine output. The analysis in step 104 may be supplemented with data from an analysis criterion database 106. The analysis criterion database 106 may comprise rules that are indicative of the threshold amount of urinary output that is necessary for a positive determination of whether there is urinary output.

The urinary output threshold may be dependent upon the type of procedure that has been performed on the patient as well as specific medications and/or fluids that the patient is currently receiving during recovery. For example, step 104 may receive data from the analysis criterion database 106 that is dependent upon the type of procedure that the patient received and sets the threshold volume of urinary output that must be achieved and the time span over which that volume of urinary output must be achieved based upon the procedure. If the threshold amount of urinary output has not been achieved over the indicated time period, an alarm 108 may be activated to notify a clinician that the patient has not produced the desired amount of urinary output. Furthermore, a record of this basic urinary output interpretation may be recorded in a patient history database 110, as it may also be desirable for a clinician to be informed that the patient has achieved the desired amount of urinary output.

After the basic urinary output interpretation 102 has been performed, the monitoring unit 14 then progresses on to the medium level of urinary output interpretation 112. The medium level 112 of urinary output interpretation is characterized by comparing the actual volume amount of urinary output to another correlated physiological parameter in the step 114 of urinary output comparison. Comparison rules from a comparison rule database 116 may be used in the urinary output comparison 114. The comparison rules may comprise specific volume ranges and time intervals over which to make the necessary urinary output comparisons. For example, the volume of the patient's urinary output over a specific time period, such as every six hours, may be analyzed to determine whether or not the urinary output is within a desired acceptable range for patient urinary output. Once again, the volume ranges and time periods from the comparison rule database depend on the type of procedure performed on the patient. The results of this comparison may be recorded in a patient history database 118 to maintain a record of the comparison data. If the patient's urinary output is out of the desired range, than an alarm 120 may be signaled to notify a clinician that the patient's urinary output is out of range. Alternatively, if the patient's urinary output is within range, then the urinary output data proceeds to the advanced level 122 of urinary output interpretation.

In an embodiment of the present invention, the advanced level interpretation 122 of patient urinary output combines the patient's urinary output with other physiological data to produce a detailed picture of patient condition based upon the relationships between the patient's urinary output and other simultaneously monitored patient physiological data. In an embodiment of the present invention, the monitoring unit 14 performs the advanced level interpretation 122, first by receiving data 124. The monitoring unit 14 receives both urinary output data 100 as well as other physiological data 126. The physiological data 126 may comprise any of the physiological data collected in the schematic diagram depicted in FIG. 1, but may include many other types of physiological data as well. Physiological data 126 may comprise the patient's ECG, blood pressure, S_{P}O₂, and expired breath carbon dioxide concentration. The physiological data 126 may also comprise current treatments or support that the patient is receiving such as intravenously supplied drugs, anesthetic, fluids in the form of saline, or other supplemental nutrition. After receiving the urinary output data and the physiological data 126, advanced rules are applied 128 to interpret the data. To apply the advanced rules 128, an advanced rule database 130 supplies the rules to be used.

The advanced rule database 130 comprises a plurality of Boolean or other logical comparisons of urinary output values and one or more other patient physiological data parameters. The Boolean logical rules may compare the values of the physiological data or urinary output in terms of data ranges combined with logical ANDs, the result of which equating a particular automated diagnosis. The Boolean logical rules may be structured in the positive or in the negative form, such that the rule either checks for the presence of a particular ailment or, alternatively, checks for the presence of normal patient physiological data ranges. In an embodiment of the present invention, the rules are structured in the positive form such that a particular patient ailment, condition, or diagnosis is characterized by a Boolean string of physiological data ranges and the rules engine 128 applies the received data 124 to this Boolean string to determine an automated diagnosis of a particular condition.

For example, the advance rule database 130 may comprise a Boolean logical rule indicating that patient hypertension is characterized by an elevated blood pressure of 5 percent or more and a decrease in urinary output by 5 percent or more and an increase in heart rate by 5 percent or more. Upon applying this Boolean logical rule from the advanced rule database 130, the rules engine 128 looks to see if this condition has been met at step 132. The results of the application of the hypertension rule are sent to a patient history database 134 so that a record of the automated patient diagnosis is maintained. If all of the conditions are not met in step 132, then the patient has not been automatedly diagnosed with hypertension The rules engine 128 may then apply the next interpretation/diagnosis rule from the advanced rule database 130. However, if all of the Boolean logical conditions have been met in step 132, then the patient is automatedly diagnosed with hypertension and an alarm 136 may be initiated to notify a clinician that an automated diagnosis of hypertension has been made.

It is understood that within the present invention, the advanced rule database 130 may comprise a variety of logical rules, and is not meant to be limited to the previous example of the Boolean logic patient hypertension diagnosis rule. The advanced rule database 130 may comprise any type of logical rule that comprises urinary output as a parameter combined with any number of one or more other physiological data 126 that may be recorded from the patient.

In an embodiment of the present invention, one of the plurality of interpretation rules in the advanced rule database 130 includes the logical combination of the patient urinary output data and a physiological parameter including data indicative of something given to the patient such as a drug, saline, or nutrition. In an embodiment of the present invention, the interpretation rule logically compares the amount of a drug delivered to the patient and the urinary output of the patient. In a further embodiment the interpretation rule logically compares the volume of saline delivered to the patient and the urinary output of the patient.

It is further understood that within the present invention, the term “alarms” need not be limited to the use of traditional visual or audio alarms that promptly notify a clinician of a detected change in patient condition. Rather, alarms as used within the present invention may also include other forms of flags or notifications that may merely appear in the patient's history or other type of digital patient record. In an embodiment of the present invention comprising the alarms being displayed in a patient history or a digital patient record, the alarms may serve as a diagnostic tool for a clinician who is notified of the automated diagnosis upon the next review of the digital patient record. The use of the alarms as a diagnostic tool would further direct the clinician's attention to the specified physiological parameters that initiated the automated diagnosis. Therefore, by the present invention, clinician monitoring of a patient's condition would be facilitated by having an additional set of digital “eyes” looking at the patient's physiological data including the patient's urinary output.

In a further embodiment of the present invention, the alarms may be further connected to a processor or controller for the automated delivery of a therapy. Therefore, upon the automated diagnosis of a patient condition, a signal may be sent from a processor or controller to a patient therapy or support device to initiate the supplying of the patient with some form of therapy or treatment, without the initiation of the therapy or treatment manually by a clinician. This automated therapy may include changes to the fluids, nutrition, or drugs delivered to the patient. For example, upon the automated diagnosis of a new patient condition, the alarm signal may be sent to a processor or controller such that a patient therapy or support device increases the delivery of saline to the patient over the next two-hour time period. The automated delivery of therapy embodiment of the present invention may be used to enhance the provision of automated patient therapy, especially, patient fluid management therapy.

It is understood that within the scope of the present invention, the monitoring unit may process the urinary output data and patient physiological data in a variety of ways. The monitoring unit 14 may comprise a CPU or a processor that may receive and process the data. Alternatively, the monitoring unit may further transmit the data to a remote CPU, processor, or server for the processing of the data before sending it back to the monitoring unit 14. It is also understood that the monitoring unit 14 may receive the urinary and patient physiological data wirelessly from the physiological parameter monitoring units.

The monitoring system of the present invention provides the distinct advantage over patient monitoring systems in the prior art as the present invention provides an improved picture of patient condition. The monitoring system of the present invention utilizes urinary output to a greater extent as a patient physiological parameter to be used for patient monitoring and automated patient diagnosis. The present invention provides not only monitoring of patient urinary output, but the recordation of this output over time and the application of increasingly more complex analysis upon the urinary output data to provide a clearer picture of patient condition with the use of the under-utilized patient physiological parameter of urinary output.

This written description uses examples to disclose the invention, including the best mode, and also to enable any person skilled in the art to make and use the invention. The patentable scope of the invention is defined by the claims and may include other examples that occur to those skilled in the art. Such other examples are intended to be within the scope of the claims if they have structural elements that do not differ from the literal language of the claims, or if they include equivalent structural elements of insubstantial difference from the literal language of the claims.

Various alternatives and embodiments are contemplated as being with in the scope of the following claims, particularly pointing out and distinctly claiming the subject matter regarded as the invention.

We claim:

1. A system for monitoring and analyzing a plurality of patient physiological parameters, the plurality of patient physiological parameters comprising patient urinary output and at least one additional physiological parameter, the apparatus comprising:
a plurality of patient connections, each patient connection disposed for collection of one or more patient parameters, one of the plurality of patient connections comprising a connection for measuring patient urinary output;
a monitoring apparatus connected to each of the patient connections, the monitoring apparatus receiving a signal from each of the patient connections indicative of a patient physiological parameter;
a processor programmed to apply at least one analysis rule to at least a signal indicative of patient urinary output;
a first database in communication with the processor, the first database comprising a first plurality of analysis rules for analyzing urinary output;
a second database in communication with the processor, the second database comprising a second plurality of rules for interpreting the signal indicative of patient urinary output and at least one signal indicative a patient parameter;
wherein the processor applies at least one of the second plurality of rules to the urinary output signal and the at least one patient parameter signal.

2. The system of claim 1, further comprising a patient history database wherein a result from the application of each analysis rule is recorded.

3. The system of claim 2 further comprising a processor programmed to produce a diagnosis of patient condition based upon the results recorded in the patient history database.

4. The system of claim 3 further comprising means for modifying the provision of an external substance to the patient in relation to the diagnosis of patient condition.

5. The system of claim 4 wherein the external substance is selected from the list comprising: nutrition, a drug, or a saline solution.

6. A method monitoring the condition of a patient, the patient connected to a plurality of patient monitoring systems, the plurality of monitoring systems comprising at least the monitoring of urine output, the method comprising:
obtaining a measurement of patient urinary output;
obtaining a measurement of at least one additional physiological parameter;
applying a basic level urinary output interpretation rule to the measurement of urinary output;
applying an advanced level urinary output interpretation rule to the measurement of urinary output and the at least one additional physiological parameter;
wherein if the basic level of urinary output interpretation rule is met, the advanced level of urinary output interpretation rule is applied and if the basic level urinary output interpretation rule is not met, an alarm signal is sent to an alarm.

7. The method of claim 6 further comprising the steps of recording the result of the application of each urinary output interpretation rule.

8. The method of claim 7 wherein the basic level urinary output interpretation rule is a time based interpretation rule.

9. The method of claim 8 wherein the time based interpretation rule is a binary determination of the measurement of urinary output over a time period.

10. The method of claim 8 further comprising applying a medium level urinary output interpretation rule to the measurement of urinary output.

11. The method of claim 10 wherein the medium level urinary output interpretation rule is a volume based interpretation rule.

12. The method of claim 11 wherein the volume based interpretation rule comprises a range of volume of desired urinary output over a specified time period.

13. The method of claim 11 wherein the advanced level urinary output interpretation rule applies a logical rule to the measurement of urinary output and the at least one additional physiological parameter.

14. The method of claim 13 wherein the at least one physiological parameter is selected from the list comprising: ECG, blood pressure, spO2, and blood gas concentration.

15. The method of claim 13 wherein the at least one physiological parameter is selected from the list comprising: administration of food, administration of a drug, and administration of saline.

16. The method of claim 6 further comprising:
displaying the result of the application of each interpretation rule;
determining a course of action in response to the result of the interpretation rule;
performing a treatment measure based upon the determined course of action.

17. The method of claim 16 wherein the treatment measure is the introduction of additional intravenous fluid.

18. The method of claim 16 wherein the treatment measure is the introduction of additional drug.

19. The method of claim 16 wherein determining a course of action is performed as part of an automated patient diagnosis system.

20. The method of claim 6 further comprising obtaining a user input, the user input indicative of specific interpretation rule to be applied to the measurement of urinary output and the measurement of at least one additional physiological parameter.

21. A method of monitoring a patient, the method comprising:
obtaining patient urinary output data;
obtaining an input from a user indicative of an analysis to perform on the patient urinary output data;
producing a first outcome indication by analyzing the urinary output data and a first parameter, the first parameter being selected from the list comprising the parameters of time and volume;
analyzing the urinary output data in light of at least one second parameter to produce a second outcome indication, the at least one second parameter being a physiological parameter;
wherein each of the outcome indications are recorded in a patient history and if one of the outcome indications is outside of a predefined value an alarm indication is initiated.
